SEQUENCE LISTING (2 sequences)

SEQ ID NO: 1 - DNA, 1026 bp; both-stranded, linear
  CDS 256..924
  CDS 430..924
tttcaccgtctagaggcataagaggtgagcccgtgctcttcagcggagaagatcccctacctggccgccggccactttctgtgggccgtggggtcctcaaggagacggcccttgggctcaggggctgcgtttccacacgcgcctttcccagggctcccgcgcccgttcctgcctggccgccggccgctccaacagcagcacaaggcgggactcagaaccggcgttcagggccgccagcggccgcgaggccctgagatgaggctccaaagaccccgacaggccccggcgggtgggaggcgcgcgccccggggcgggcggggctccccctaccggccagacccggggagaggcgcgcggaggctgcgaaggttccagaagggcggggagggggcgccgcgcgctgaccctccctgggcaccgctggggacgatggcgctgctcgccttgctgctggtcgtggccctaccgcgggtgtggacagacgccaaccygactgcgagacaacgagatccasaggactcccagcgaacggacgagggtgacaatagagtgtggtgtcatgtttgtgagagagaaaacactttcgagtgccagaacccaaggaggtgcaaatggacagagccatactgcgttatagcggccgtgaaaatatttccacgttttttcatggttgcgaagcagtgctccgctggttgtgcagcgatggagagacccaagccagaggagaagcggtttctcctggaagagcccatgcccttcttttacctcaagtgttgtaaaattcgctactgcaatttagaggggccacctatcaactcatcagtgttcaaagaatatgctgggagcatgggtgagagctgtggtgggctgtggctggccatcctcctgctgctggcctccattgcagccggcctcagcctgtcttgagccacgggactgccacagactgagccttccggagcatggactcgctccagaccgttgtcacctgttgcattaaacttgttttctgttgaaaaaaaaaaa

SEQ ID NO: 2 - protein, 223 aa
MRLQRPRQAPAGGRRAPRGGRGSPYRPDPGRGARRLRRFQKGGEGAPRADPPWAPLGTMALLALLLVVALPRVWTDANXTARQRDPXDSQRTDEGDNRVWCHVCERENTFECQNPRRCKWTEPYCVIAAVKIFPRFFMVAKQCSAGCAAMERPKPEEKRFLLEEPMPFFYLKCCKIRYCNLEGPPINSSVFKEYAGSMGESCGGLWLAILLLLASIAAGLSLS

(19) United States
(12) Patent Application Publication
(10) Pub. No.: US 2003/0022858 A1
(43) Pub. Date: Jan. 30, 2003

(54) Fanconi-gen II

(76) Inventors: Manfred Kubbies, Penzberg (DE); Andreas Machl, Penzberg (DE); Simone Planitzer, Muenchen (DE)

Correspondence Address: HOFFMANN-LA ROCHE INC., PATENT LAW DEPARTMENT, 340 KINGSLAND STREET, NUTLEY, NJ, 07110

(21) Appl. No.: 10/199,448

(22) Filed: Jul. 19, 2002

Related U.S. Application Data
(63) Continuation of application No. 09/977,801, filed on Oct. 15, 2001.
Application No. 09/977,801 is a continuation of application No. 09/402,632, filed on Dec. 17, 1999, now abandoned.

(30) Foreign Application Priority Data
Apr. 7, 1997 (EP) 97105688.2
Apr. 6, 1998 (WO) PCT/EP98/01994

(51) Int. Cl. A61K 48/00; C07H 21/04; C12N 9/00; C12P 21/02; C12N 5/08

(52) U.S. Cl. 514/44; 536/23.2; 435/183; 435/69.1; 435/320.1; 435/372

(57) ABSTRACT

The present invention concerns a pathophysiologically relevant gene associated with Fanconi anaemia (FA), a polypeptide coded thereby, an antibody directed against the polypeptide as well as the pharmaceutical applications of the nucleic acid, polypeptide and antibody.

### DESCRIPTION

The present invention concerns a pathophysiologically relevant gene associated with Fanconi anaemia (FA), a polypeptide coded thereby, an antibody directed against the polypeptide as well as the pharmaceutical use of the nucleic acid, polypeptide and antibody.

FA is a rare genetic disease which is characterized by progressive pancytopenia, congenital abnormalities and an increased risk of tumour diseases (Glanz and Fraser, J.Med.Genet. 19 (1982) 412-416; Auerbach and Allen, Cancer Genet.Cytogenet. 51 (1991) 1-12). It occurs in about one in 300,000 persons.

Although the molecular basis of the disease is unknown, the hypersensitivity towards the clastogenic effect of DNA cross-linking agents is a good marker for the FA genotype (Auerbach and Wolmann, Nature 261 (1976), 494-496). Cells from FA patients exhibit multiple chromatid breaks and chromatid substitutions after contact with mytomycin C (MMC) or diepoxybutane (DEB) at concentrations which have a low clastogenic effect on normal cells (Sasaki and Tonomura, Cancer Res. 33 (1973), 1829-1836 and Auerbach, Exp. Hematol. 21 (1993), 731). A defect in the G2 phase of the cell cycle becomes apparent after incubation of FA lymphocytes and fibroblasts with MMC which is manifested by a delay in the G2 phase transition as well as in a complete arrest (Kubbies et al., Am.J.Hum.Genet. 37 (1985), 1022; Hoehn et al., Fanconi Anaemia, Clinical, Cytogenic and Experimental Aspects (1989), Springer Verlag Berlin-Heidelberg; Seyschab et al., Blood 85 (1995), 2233-2237).

At present at least 5 different complementation groups are known within the FA population (Duckworth-Rysiecki et al., Somatic Cell Mol.Genet. 11 (1985), 35; Strathdee et al., Nature 356 (1992), 763; Joenje et al., Blood 86 (1995), 2156-2160). The genes for the complementation groups A and C have recently been described (Strathdee et al., Nature 356 (1992), 763; Lo Ten Fol et al., Nature Genet. 14 (1996), 320-323); WO93/22435; Pronk et al., Nature Genet. 11 (1995), 338-340) but the type of molecular mechanism of action of the FA-A and FA-C proteins is still unknown (Gavish et al., Am.J.Hum. Genet. 53 (1993), 685; Yamashita et al., Proc.Natl.Acad. Sci. USA 91 (1994), 6712; Youssoufian et al., J.Biol. Chem. 270 (1995), 9876-9882). Furthermore the chromosomal location has been specified for the complementation group FAD (Whitney et al., Nature Genet. 11 (1995), 341-343).

The object of the present invention was to identify new genes which are involved in the DNA regulation cascade (e.g. cell cycle disorders, DNA repair, tumorigenesis/tumour progression) and which may be associated with the pathophysiological phenotype of Fanconi anaemia.

The present invention describes the identification, cloning and characterization of a gene which is named the Fanconi gene II and codes for two new polypeptides. This gene sequence was found using the differential display technique (Liang and Pardee, Science 257 (1992), 967-971) in a comparison of normal fibroblasts and FA fibroblasts. The Fanconi gene II is not expressed in FA fibroblasts but in normal fibroblasts. The Fanconi gene II, the polypeptides coded thereby as well as antibodies directed against the polypeptides are suitable as diagnostic, therapeutic or preventive agents for diseases that are directly or indirectly associated with disorders of the cell cycle, cell activation, cell cycle progression, DNA repair, cytopenias, tumorigenesis and tumour progression.

A subject matter of the present invention is a nucleic acid which comprises

(a) the nucleotide sequence shown in SEQ ID NO. 1 or a protein-coding section thereof,

(b) a nucleotide sequence corresponding to the sequence from (a) within the scope of the degeneracy of the genetic code or

(c) a nucleotide sequence hybridizing with the sequences from (a) or/and (b) under stringent conditions.

Three cDNA sequences W44613, W44574, g1664579, 1996 are specified in the EMBL EST data bank which contain sections of the nucleotide sequence are shown in SEQ ID No. 1. These sequences are not a subject matter of the invention. They disclose neither the complete nucleic acid sequence according to the invention nor a functional protein-coding section thereof since no open reading frame is disclosed due to the lack of a start codon in all three sequences. Furthermore the three sequences have no 5′-UTRs and contain deletions which result in a shift of the reading frame. Moreover no biological function is disclosed for the three aforementioned sequences.

The nucleotide sequence shown in SEQ ID NO. 1 contains two open reading frames which correspond to polypeptides with a length of 223 amino acids and 165 amino acids. These polypeptides extend from amino acid 1 to 223 or from amino acid 59 to 223 of the amino acid sequence shown in SEQ ID NO. 2.

In SEQ ID NO. 1 a nucleotide Y i.e. C or T is present at position 491 and a nucleotide S i.e. C or G is present at position 514.

In addition to the nucleotide sequence shown in SEQ ID NO. 1 and a nucleotide sequence corresponding to this sequence within the scope of the degeneracy of the genetic code, the present invention also concerns a nucleotide sequence which hybridizes with one of the aforementioned sequences. In the present invention the term “hybridization” is used as in Sambrook et al. (Molecular Cloning. A Laboratory Manual, Cold Spring Harbor Laboratory Press (1989), 1.101-1.104). A stringent hybridization preferably means that a positive hybridization signal is still observed after washing for one hour with 1×SSC and 0.1% SDS at 50° C., preferably at 55° C., particularly preferably at 62° C. and most preferably at 68° C. and in particular for one hour in 0.2×SSC and 0.1% SDS at 55° C., preferably at 55° C., particularly preferably at 62° C. and most preferably at 68° C. A nucleotide sequence hybridizing under such washing conditions with the nucleotide sequence shown in SEQ ID NO. 1 or a corresponding nucleotide sequence within the scope of the degeneracy of the genetic code is a nucleotide sequence according to the invention.

The nucleotide sequence according to the invention is preferably a DNA. It can, however, also comprise an RNA or a nucleic acid analogue such as a peptidic nucleic acid. The nucleic acid according to the invention particularly preferably contains a protein-coding section of the nucleotide sequence shown in SEQ ID NO. 1 or a sequence which has a homology of more than 80%, preferably of more than 90% and particularly preferably of more than 95% to the nucleotide sequence shown in SEQ ID NO. 1 or a preferably at least 20 nt and particularly preferably at least 50 nt long section thereof.

A further subject matter of the present invention are the polypeptides coded by a nucleic acid as stated above. These polypeptides preferably have (a) the amino acid sequence shown in SEQ ID NO. 2 of amino acids 1 to 223, (b) the amino acid sequence shown in SEQ ID NO. 2 of amino acids 59 to 223 or (c) a homology of more than 70%, preferably of more than 80% and particularly preferably of more than 90% to one of the amino acid sequences according to (a) or (b).

Nucleic acids according to the invention are preferably obtainable from mammals and in particular from humans. They can be isolated by known techniques using short sections of the nucleotide sequence shown in SEQ ID NO. 1 as hybridization probes or/and primers according to known methods. Furthermore nucleic acids according to the invention can also be prepared by chemical synthesis in which case modified nucleotide building blocks e.g. 2′-O-alkylated nucleotide building blocks can optionally be used instead of the usual nucleotide building blocks. Nucleic acids which are partially or completely composed of modified nucleotide building blocks can for example be used as therapeutic agents e.g. as antisense nucleic acids or ribozymes.

The invention also encompasses nucleic acid analogues such as peptidic nucleic acids whose base sequence corresponds to a nucleic acid according to the invention.

A further subject matter of the present invention is a vector which contains at least one copy of a nucleic acid according to the invention. This vector can be any desired prokaryotic or eukaryotic vector on which the DNA sequence according to the invention is located preferably under the control of an expression signal (promoter, operator, enhancer etc.). Examples of prokaryotic vectors are chromosomal vectors such as bacteriophages and extrachromosomal vectors such as plasmids, circular plasmid vectors being particularly preferred. Suitable prokaryotic vectors are described for example in Sambrook et al., Supra, chapters 1- 4.

The vector according to the invention is particularly preferably a eukaryotic vector e.g. a yeast vector or a vector suitable for higher cells (e.g. a plasmid vector, viral vector, plant vector). Such vectors are known to a person skilled in the field of molecular biology and do not therefore need to be elucidated in more detail here. In this connection particular reference is made to Sambrook et al., Supra, chapter 16.

In addition to the polypeptides shown in SEQ ID NO. 2, the invention also concerns muteins, variants and fragments thereof. These are understood as sequences which differ from the amino acid sequences shown in SEQ ID NO. 2 by substitution, deletion or/and insertion of individual amino acids or short sections of amino acids.

The term “variant” includes naturally occurring allelic variations or splice variations of the Fanconi poly-peptide II as well as proteins produced by recombinant DNA technology (in particular by in vitro mutagenesis with the aid of chemically synthesized oligonucleotides) which essentially correspond to the protein shown in SEQ ID NO. 2 with regard to their biological or/and immunological activity. This term also includes chemically modified polypeptides. These include polypeptides whose termini or/and reactive amino acid side groups have been modified by acylation e.g. acetylation or amidation.

The invention also concerns a vector which contains an at least 20 nucleotide long section of the sequence shown in SEQ ID NO. 1. This section preferably has a nucleotide sequence which is derived from the protein-coding region of the sequence shown in SEQ ID NO. 1 or from a region that is essential for the expression of the protein. These nucleic acids are particularly suitable for the production of antisense nucleic acids that can be used therapeutically which preferably are up to 50 nucleotides long.

A further subject matter of the present invention is a cell which is transformed with a nucleic acid according to the invention or with a vector according to the invention. The cell can be a eukaryotic as well as a prokaryotic cell. Methods for transforming cells with nucleic acids are general state of the art and therefore do not need to be elucidated in more detail. Examples of preferred cells are eukaryotic cells, in particular animal cells and particularly preferably mammalian cells.

A further subject matter of the present invention is the use of the polypeptide according to the invention or fragments of this polypeptide as an immunogen for the production of antibodies. In this case antibodies can be produced in the usual manner by immunizing experimental animals with the complete polypeptide or fragments thereof and subsequently isolating the resulting polyclonal antisera. Monoclonal antibodies can be obtained in a known manner from the antibody-producing cells of the experimental animals by cell fusion according to the method of Köhler and Milstein or further developments thereof. Human monoclonal antibodies can also be produced by known methods.

The recombinant Fanconi II proteins or peptide fragments, in particular N-terminal or C-terminal peptides thereof, are preferred as the immunogen.

Hence a further subject matter of the present invention is an antibody to the Fanconi II proteins or variants thereof, preferably antibodies which exhibit no cross-reaction with other Fanconi-associated proteins such as the FAC protein. The antibodies are particularly preferably directed against the entire polypeptides or against a peptide sequence which corresponds to the amino acids 1-40, 59-120 or 205-223 of the amino acid sequence shown in SEQ ID NO. 2.

The provision of Fanconi II proteins, nucleic acids coding therefor and antibodies directed against them are a prerequisite for a specific search for effectors of these proteins. Substances which have an inhibitory or activating effect on the polypeptide according to the invention are able to selectively influence the cell functions controlled by the polypeptide. Consequently they can be used to treat corresponding clinical pictures such as e.g. cytopenias or tumours. Hence a subject matter of the invention is also a method for identifying effectors of the Fanconi II proteins in which cells that express the protein are contacted with various potential effector substances e.g. low molecular substances and the cells are analysed for changes e.g. changes leading to cell activation, cell inhibition, cell proliferation or/and genetic changes in the cells. Binding targets of the Fanconi II proteins can also be identified in this manner.

In the case of clinical pictures which are due to a defect in the Fanconi II proteins it is possible to carry out a gene therapy which comprises the transfer of a nucleic acid coding for the Fanconi II proteins into the appropriate target tissue by means of vectors e.g. viral vectors. On the other hand disease states which are due to an uncontrolled expression of the Fanconi II proteins can be treated by a gene therapy which blocks this expression.

Moreover the results presented also provide the basis for a targetted diagnosis of diseases which are causally or indirectly linked to changes in the activity of the Fanconi II proteins. These examinations can be carried out with the aid of specific nucleic acid probes for detection at the nucleic acid level e.g. at a gene or transcript level or with the aid of antibodies to the Fanconi II proteins for detection at the polypeptide level.

Hence the present invention concerns a pharmaceutical composition which contains nucleic acids, vectors, cells, polypeptides and antibodies as stated above as active components.

The pharmaceutical composition according to the invention can also contain common pharmaceutical carrier substances, auxiliary substances or/and additives as well as optionally further active components. The pharmaceutical composition can be used in particular for the diagnosis, treatment or prevention of diseases which are associated with disorders of the cell cycle, cell activation, cell cycle progression, DNA repair and with cytopenias, tumorigenesis or/and tumour progression. Furthermore the composition according to the invention can also be used to diagnose a predisposition for such diseases individuals, in particular to diagnose a risk for cytopenias or/and tumour diseases.

Yet a further subject matter of the present invention is a method for diagnosing the above-mentioned diseases in which a patient or a sample, such as a sample of a body fluid or of a tissue, derived from a patient is contacted with a pharmaceutical composition according to the invention and the nucleotide sequence or/and the expression of the nucleic acid according to the invention is determined qualitatively or quantitatively. These methods of determination can for example be carried out at the nucleic acid level by using nucleic acid hybridization probes or by means of reverse transcription/PCR, or at the protein level by antibodies using cytochemical or histochemical methods. The pharmaceutical composition is particular preferably used as a marker for the occurrence of cytopenias, tumours or other proliferation-associated diseases or of a predisposition for the said pathophysiological changes.

Finally the present invention also concerns a method for the treatment or prevention of one of the aforementioned diseases in which a pharmaceutical composition according to the invention is administered to a patient which contains the active component in an amount that is effective against the disease. Specific examples of pharmaceutical compositions which are suitable for therapeutic purposes are for example bispecific antibodies and antibody-toxin or antibody-enzyme conjugates. Further preferred pharmaceutical compositions for therapeutic purposes are antisense nucleic acids, gene therapeutic vectors or other low molecular activators or inhibitors.

The invention is further elucidated by the following examples and the sequence protocol.

SEQ ID NO. 1 shows a nucleotide sequence which contains the genetic information coding for the Fanconi gene II in which a larger open reading frame extends from nucleotide 256-924 and a smaller open reading frame from nucleotide 430-924, and

SEQ ID NO. 2 shows the amino acid sequences of the open reading frames of the nucleotide sequence shown in SEQ ID NO. 1 in which the amino acid sequence of the larger open reading frame extends from amino acid 1-223 and the amino acid sequence of the smaller open reading frame extends from amino acid 59-223.

### EXAMPLES

### Example 1

### Cell Culture

Primary diploid human fibroblasts H94-38 and H94-17 were isolated from foetal lung tissue and provided by D. Schindler (University of Würzburg). The H94-38 cells were diagnosed by cell cycle analysis as the Fanconi anaemia phenotype and have an extended G2 phase as well as an increase of the G2 phase arrest when MMC is added. Complementation investigations show that the H94-38 cells do not belong to the Fanconi complementation groups A, B, C and D but presumably to the complementation group E. H94-17 control cells exhibit no increase in MMC sensitivity.

The cells were cultured at 37° C. and with 7% CO₂ and 95% humidity in MEM medium containing Earle's salts (BRL, Gaithersburg Md., U.S.) to which 10% foetal calf serum (Hyclone, Logan, Utah, U.S.A.) was added. For the RNA preparation the cells were synchronized by serum withdrawal (0.1%) and stimulated after 48 h with 10% foetal calf serum. After a further 30 h the cells were subconfluent and could be harvested for the RNA isolation.

After a culture period of 30 h in medium containing BrdU aliquots of these cell cultures were taken for analysis of the cell cycle status in a proliferation assay. The number of cells in the cell cycle phases G0/G1, S and G2/M were determined as described by Kubbies (in Radbruch, A. (ed.) Flow Cytometry and Cell Sorting, Springer Verlag Berlin-Heidelberg 1992, pp 75-85) by means of a high resolution flow cytometric BrdU Hoechst quenching technique.

### Example 2

### mRNA Differential Display

The RNA kit from Gen Hunter (Brookline, Mass., U.S.A.) was used for the mRNA differential display. The total RNA was isolated from synchronized cell cultures using the Tripure reagent (Boehringer Mannheim GmbH, GER) according to the manufacturer's instructions. The RNA was stored until use at −80° C. as isopropanol-precipitated RNA pellets covered with 70% ethanol. DNAse I (Boehringer Mannheim GmbH) was added to 1-5 μg total RNA in 1×DNAse I reaction buffer and incubated for 30 min at 37° C.

The RNA samples were determined quantitatively by measuring the absorbance at 260 nm and analysed on an agarose gel. 0.2 μg total RNA was used for the reverse transcription. A total of 8 μg total RNA was isolated from 1×10⁶ fibroblasts.

The reverse transcription of the RNA was carried out in double reaction mixtures of 20 μl in each case in 1×reverse transcription buffer, 20 μm of each DNTP and 2 μM of each of the single base anchor primers T₁₁A, T₁₁G or T₁₁C. The solution was heated for 5 min to 65° C., cooled for 10 min to 37° C. and then 100 U Moloney murine leukaemia virus (MMLV) reverse transcriptase was added. After incubating for 1 hour at 37° C. the mixture was heated for 5 min to 75° C. and then stored at −20° C.

The PCR was carried out in a reaction solution which contained 1/10 volumes of the mixture for reverse transcription, 2 μM dNTPs, 0.2 μM of the respective T₁₁N primer, 0.2 μM of a primer with an arbitrarily determined sequence, 10 μCi α[³⁵S] dATP and 1 U Taq-DNA polymerase (Boehringer Mannheim GmbH). The PCR was carried out in a Perkin-Elmer 2400 Gene Amp. PCR system for a total of 40 cycles of 30 sec at 94° C., 2 min at 40° C., 30 sec at 72° C. and finally 5 min at 72° C. Various arbitrary primers from Gen Hunter (13-mer with HindIII restriction site), Operon (Alameda Calif., U.S.A.) and Genosys (The Woodlands, Tex., U.S.A.) (in each case 10mer primers with 60-70% GC content) were used.

The samples were denatured in sequencing gel loading buffer at 80° C. for 2 min before separation on a 5-6% denaturing polyacrylamide sequencing gel. Double PCR experiments were carried out on each sample and they were separated next to one another on the same polyacrylamide gel. The dried gel was analysed by autoradiography for differentially expressed genes.

Reproducible bands which correspond to differentially expressed genes were cut out of the gel. The cDNA was eluted from the gel pieces by boiling for 15 min in 100 μl sterile water. The DNA in the supernatant was collected by ethanol precipitation in the presence ff glycogen. Subsequently the corresponding primers and PCR conditions as described above were used to reamplify the DNA except that DNTP concentrations of 20 μM were used and the reaction mixture contained no radioisotopes.

The amplified PCR fragments obtained in this manner were separated on an agarose gel and eluted by centrifugation of the corresponding gel piece in a 0.45 μm Millipore Durapore membrane tube. The samples were stored at −20° C. for Northern analysis.

In this manner a total of 60 bands, 43 of which were reamplified bands were obtained from 106 different primer combinations which correspond to differentially expressed genes in FA cells and control cells. This differential expression was reproducible.

### Example 3

### Northern Analysis

The Northern blot analysis was carried out by standard procedures according to Sambrook et al. (1989), Supra. The nucleic acids were transferred onto positively-charged nylon membranes (Boehringer Mannheim GmbH) by downwards directed capillary transfer using 10×SSC and cross-linked.

Specific probes were directly labelled from the PCR reamplification mixture by labelling with the Hi-Prime labelling kit (Boehringer Mannheim GmbH) using hexamer primers with an arbitrary sequence. Free nucleotides were separated using G50 Sephadex spin columns (Boehringer Mannheim GmbH).

The probes produced in this manner were hybridized with total RNA. After hybridization for 16-20 h at 42° C., the filters were washed twice in 1×SSC, 0.1% SDS at room temperature for 15 min and subsequently in 1×SSC 0.1% SDS at 50° C. for 1 h. Then the membranes were examined by autoradiography.

The Northern analysis showed a differential expression for an approximately 1020 bp long PCR fragment.

Northern analyses in cell culture and tissue samples showed in control fibroblasts a dominant band with a length of ca. 1 kb and often a further band or singly occurring band with a length of ca. 700 bp which may perhaps be due to a splice variant. These bands were not found in the examined FA fibroblasts. Both variants were strongly expressed in the tumour cell line HeLa. No expression was found in other tumour cell lines (e.g. Raji or K562). Only the larger band was found in embryonic fibroblasts from the cartilage of the eye pigment shell.

### Example 4

### Characterization of DD-PCR Fragments which Correspond to Differentially Expressed mRNA Species

PCR fragments which were shown to be differential in the Northern blot were ligated by means of the TA cloning kit (INVITROGEN) into the vector pCR™2.1 according to the manufacturer's instructions and the E. coli strain INVαF′ was transformed with this construct. Clones which contained the plasmid composed of differential fragment and vector were cultured by standard procedures according to Sambrook et al. (1989, Cold Spring Harbor University Press, Cold Spring Harbor, N.Y.) and the plasmids were isolated.

The 5′region of the nucleotide sequence that was found was amplified by a modified new RACE technique (Frohmann, M. A. (1994). For this a DNA/RNA oligonucleotide (5′-GTAAAACGACGGCCAGTAAAGCACTCTCCAGCCTCTCACCGCrArArA-3-3′) was ligated to the 5′end the total RNA of the control cells in the presence of 20% PEG/DMSO (1:1, w/v) and the modified RNA was reversely transcribed with the specific primer SP1 (5′-AACAGAAAACAAGTTTAATGCAACAGGTGA-3′). The total cDNA transcript was amplified with a primer (5′-CACTCTCCAGCCTCTCACCGCAAA-3) and with the gene-specific primer SB2 (5′-GCTGAGGCC GGCTGCAATGGA-3) and ligated as described above into the vector pCR™2.1 and sequenced. The fact that both fragments belong to the same mRNA was proven by an overlapping region of 380 nucleotides with an identical base sequence and a PCR with primers located outside at the 5′end of the RACE fragment (5′-TTTCACCGTCTAGAGGCATAAGAGG-3′) and at the 3′end of the differential display fragment (5′-AACAGAAAACAAGTTTAATGCAACAGGTGA-3′) which resulted in the sequence of the Fanconi gene II. The cDNA obtained in this manner of the Fanconi gene II was ligated as described above into the vector pCR™2.1 and sequenced. The differential expression of the entire Fanconi gene II mRNA was demonstrated by means of Northern blot analysis as described in example 3.

### Example 5

### Preparation of an Expression Construct and Expression

The expression of the long variant of the FA-II gene is described here as an example. However, the method can also be applied to the shortened form of the FA-II gene. The FA-II gene described in example 4 which codes for amino acid sequence 1-223 of SEQ ID NO. 2, was recloned by standard methods (Sambrook, Fritsch, Maniatis, Molecular Cloning; A Laboratory Manual, 2nd Edition, Cold Spring Harbor, 1989) into the eukaryotic expression vector pCDNA3 (Invitrogen). The expression was regulated by the CMV promoter/enhancer and the BGH polyA signal. The Neo gene is used as the selection gene (under the control of the SV40 expression cassette).

CHO cells were used to prepare a stable cell line expressing FA-II. For this 20 μg lipofectamin (Gibco; in 750 μl MEM-alpha medium) was mixed with 10 μg DNA (in 750 μl MEM-alpha medium), incubated for 45 minutes at room temperature and subsequently diluted with 6 ml MEM-alpha medium. This mixture was added for 6 hours to 5×10⁶ CHO cells in T75 cell culture bottles (Nunc) in MEM-alpha medium (Gibco). The incubation was carried out at 37° C. The cells were subsequently washed with MEM-alpha/10% FCS (foetal calf serum) and cultured for 48 hours at 37° C. in fresh medium. Subsequently selection pressure was applied by adding 1 mg/ml neomycin (G418, Boehringer Mannheim). The surviving cells were cloned by means of FACS (Becton Dickinson) as single cells in 96-well culture plates (Nunc) containing fresh medium and further cultured under G418 selection pressure until a stably transfected CHO clone had been established.

By using DHFR as a selection gene it is possible to achieve a gene amplification of the FA-II gene in addition to obtaining a stably transfected CHO cell line.

### Example 6

### Antibody Production

As already described in example 5 the antibody production is described here using the long variant of the FA-II protein as an example. The method also applies to the shortened form of the FA-II protein.

BALB/c mice were immunized intraperitoneally with recombinant, human FA-II protein with the amino acid sequence 1-223 of SEQ ID NO. 2 (prepared in CHO cells). The primary immunization was carried out in complete Freund's adjuvant and all further immunizations were carried out in incomplete Freund's adjuvant. The dose was 50-100 μg. Subsequent immunizations were carried out at ca. .4-week intervals until a serum titre of 1:50,000 is reached.

Subsequently the spleen cells of the immunized animals were immortalized with the myeloma cell line P3XX63.Ag8.653. The fusion was carried out according to standard methods (J. Immunol. Methods 39 (1980), 285-308). The fusion ratio of spleen cells to myeloma cells was 1:1. The fusion products were sown out on 24-well cell culture dishes (Nunc) in HA medium based on RPMI/10% FCS (Boehringer Mannheim). 2 Weeks after fusion positive primary cultures were cloned as individual cells in 96-well cell culture plates (Nunc) in·RPMI/10% FCS by means of FACS (Becton Dickinson).

In order to obtain monoclonal antibodies the hybridoma cell clones obtained in this manner were expanded in vivo. For this 5×10⁶ hybridoma cells were inoculated intraperitoneally into mice that had been pretreated with Tristan (Sigma Chemical Company). After 10-21 days 2-3 ml ascites was withdrawn from each mouse and the monoclonal antibody was isolated therefrom by conventional methods.

1. Nucleic acid,
wherein
it comprises
(a) the nucleotide sequence shown in SEQ ID NO. 1 or a protein-coding section thereof,
(b) a nucleotide sequence corresponding to the sequence from (a) within the scope of the degeneracy of the genetic code or
(c) a nucleotide sequence hybridizing with the sequences from (a) or/and (b) under stringent conditions,
provided that the nucleic acid is different from the nucleotide sequences with the accession numbers W44613, W44574 and g1664579 specified in the EMBL EST data bank.

2. Nucleic acid as claimed in claim 1,
wherein
it comprises a protein-coding section of the nucleotide sequence shown in SEQ ID NO. 1.

3. Nucleic acid as claimed in claim 1,
wherein
it has a homology of more than 80% to the nucleotide sequence shown in SEQ ID NO. 1 or to a section thereof.

4. Modified nucleic acid or nucleic acid analogue which comprises a nucleotide sequence as claimed in one of the claims 1-3.

5. Vector,
wherein
it contains at least one copy of a nucleic acid as claimed in one of the claims 1 to 3 or a section thereof.

6. Vector as claimed in claim 5,
wherein
it enables the expression of the nucleic acid in a suitable host cell.

7. Cell,
wherein
it is transformed with a nucleic acid as claimed in one of the claims 1 to 3 or with a vector as claimed in claim 5 or 6.

8. Polypeptide,
wherein
it is coded by a nucleic acid as claimed in one of the claims 1 to 3, whereby the provision of claim 1 does not have to be taken into consideration.

9. Polypeptide as claimed in claim 8,
wherein
it has
(a) the amino acid sequence from 1-223 shown in SEQ ID NO. 2,
(b) the amino acid sequence from amino acid 59-223 shown in SEQ ID NO. 2 or
(b) a homology of more than 70% to one of the amino acid sequences according to (a) or (b).

10. Use of a polypeptide as claimed in claim 8 or 9 or of fragments of this polypeptide as an immunogen for the production of antibodies.

11. Antibody against a polypeptide as claimed in claim 8 or 9.

12. Antibody as claimed in claim 11,
wherein
it is directed against the entire polypeptide or against a peptide sequence corresponding to the amino acids 1-40, 59-120 or 205-223 from SEQ ID NO. 2.

13. Modified polypeptide which comprises an amino acid sequence as claimed in 8 or 9.

14. Pharmaceutical composition,
wherein
it comprises as the active component
(a) a nucleic acid claimed in one of the claims 1 to 4, whereby the provision of claim 1 does not have to be taken into consideration,
(b) a vector as claimed in claim 5 or 6,
(c) a cell as claimed in claim 7,
(d) a polypeptide as claimed in claim 8, 9 or 13 or/and
(e) an antibody as claimed in claim 11 or 12.

15. Composition as claimed in claim 14,
wherein
it additionally contains common pharmaceutical carrier substances, auxiliary substances or/and additives.

16. Use of a composition as claimed in claim 14 or 15 for diagnosing diseases which are associated with disorders of the cell cycle, cell activation, cell cycle progression, DNA repair, cytopenias, tumorigenesis or/and tumour progression.

17. Use of a composition as claimed in claim 14 or 15 for diagnosing a predisposition to diseases that are associated with disorders of the cell cycle, cell activation, cell cycle progression, DNA repair, cytopenias, tumorigenesis or/and tumour progression.

18. Use of a composition as claimed in claim 14 or 15 for the production of an agent for diagnosing diseases that are associated with disorders of the cell cycle, cell activation, cell cycle progression, DNA repair, cytopenias, tumorigenesis or/and tumour progression or an agent for diagnosing a predisposition to such diseases.

19. Use of a composition as claimed in claim 14 or 15 for the production of an agent for the treatment or prevention of diseases that are associated with disorders of the cell cycle, cell activation, cell cycle progression, DNA repair, cytopenias, tumorigenesis or/and tumour progression.

20. Use as claimed in claim 19 for the production of an agent for gene therapy.

21. Method for diagnosing diseases that are associated with disorders of the cell cycle, cell Activation, cell cycle progression, DNA repair, cytopenias, tumorigenesis or/and tumour progression or with a predisposition to such diseases,
wherein
a patient or a sample derived from a patient is contacted with a composition as claimed in claim 14 or 15 and the nucleotide sequence or/and the expression of a nucleic acid as claimed in claim 1 is determined, whereby the provision of claim 1 does not have to be taken into consideration.

22. Method for the treatment or prevention of diseases that are associated with disorders of the cell cycle, cell activation, cell cycle progression, DNA repair, cytopenias, tumorigenesis or/and tumour progression,
wherein
a composition as claimed in claim 14 or 15 which contains the active component in an amount that is effective against such a disease is administered to a patient.

23. Method for the identification of effectors of a protein as claimed in claim 8 or 9,
wherein
cells which express the protein are contacted with various potential effector substances and the cells are analysed for changes.
